(19) United States
(12) Patent Application Publication
(10) Pub. No.: US 2020/0139334 A1
(43) Pub. Date: May 7, 2020

(54) Method for Temperature Control in a Bubble Column Reactor for Selective 1-Hexene Production

(71) Applicant: SABIC GLOBAL TECHNOLOGIES B.V., BERGEN OP ZOOM (NL)

(72) Inventors: Abdullah Saad Al-Dughaiter, Riyadh (SA); Shahid Azam, Riyadh (SA); Abdulmajeed Mohammed Al-Hamdan, Riyadh (SA); Dafer Mubarak Alshahrani, Riyadh (SA); Ralf Noack, Dresden (DE); Tobias Meier, Dresden (DE); Gabriel Waurick, Dresden (DE); Heinz Bolt, Wolfratshausen (DE); Anina Wohl, München (DE); Wolfgang Müller, München (DE); Andreas Meiswinkel, Rimsting (DE); Hans-Jorg Zander, München (DE)

(21) Appl. No.: 16/473,537

(22) PCT Filed: Dec. 21, 2017
(86) PCT No.: PCT/IB2017/058306

§ 371 Date: Jun. 25, 2019

Related U.S. Application Data
(60) Provisional application No. 62/440,431, filed on Dec. 30, 2016.

(51) Int. Cl. B01J 19/00; C07C 2/08

(52) U.S. Cl. CPC B01J 19/0013; B01J 2219/0013; C07C 2/08

(57) ABSTRACT

A method of temperature control includes: passing a feed stream comprising ethylene through a reactor at a feed location; withdrawing an outlet steam comprising linear alpha olefins from the reactor; passing the outlet stream through a condensate vessel, wherein the outlet stream is split into a vapor fraction and a liquid fraction within the condensate vessel; withdrawing the vapor fraction from the condensate vessel and recycling it back to the feed stream; and withdrawing the liquid fraction from the condensate vessel and injecting it into the reactor at an injection location.

### BACKGROUND

The commercial processes for ethylene trimerization involve feeding a solvent such as toluene, ethylene recycle with fresh ethylene makeup, and a respective catalyst solution into a reactor, e.g., a multi-tubular reactor, such as a bubble column reactor. Un-reacted ethylene and light ends linear alpha olefins that have partitioned into vapor phase exit from the top of the reactor. The bottom reactor effluent contains the linear alpha olefins products together with dissolved ethylene, solvent, and catalyst, and are continuously withdrawn from the bottom of the reactor.

Bubble column reactors are generally utilized to make linear alpha olefins. For example, bubble column reactors are utilized in an oligomerization process of ethylene to provide linear alpha olefins. Such a bubble column reactor comprises a bottom compartment for introducing a gaseous monomer feed and separated from an upper reaction compartment. In the upper reaction compartment, the bubble column reactor includes a lower 2-phase section and an upper gaseous phase section. Typically, the column reactor is operated continuously, as a monomer feed, solvent, and catalyst are continuously supplied and the solvent, linear alpha olefin and catalyst are continuously removed.

The trimerization reaction of ethylene into 1-hexene is a highly exothermic reaction with the heat of reaction approximately 25 kcal per converted mole of ethylene. The released heat has to be removed to maintain the required reactor temperature.

Thus, there is a need for a method of removing the heat of reaction during the selective production of 1-hexene in a bubble column reactor.

### SUMMARY

Disclosed, in various embodiments, are methods for temperature control in a bubble column reactor, where highly exothermic reaction of ethylene trimerization to 1-hexene is occurring.

A method of temperature control includes: passing a feed stream comprising ethylene and catalyst through a reactor at a feed location; withdrawing an outlet steam comprising linear alpha olefins from the reactor, wherein the outlet stream is taken from a vapor phase within the reactor; passing the outlet stream through a condensate vessel, wherein the outlet stream is split into a vapor fraction and a liquid fraction within the condensate vessel; withdrawing the vapor fraction from the condensate vessel, optionally passing the vapor fraction through a purge stream, and recycling the vapor fraction back to the feed stream; and withdrawing the liquid fraction from the condensate vessel and injecting it into the reactor at an injection location.

A method of temperature control includes: passing a feed stream comprising ethylene and a liquid solvent through a reactor at a feed location, wherein the reactor is a two-phase bubble column reactor comprising a liquid phase and a vapor phase, wherein an oligomerization reaction, preferably a trimerization reaction occurs within the reactor; withdrawing an outlet steam comprising unconverted ethylene, butene-1, solvent, optionally other and/or higher carbon number linear alpha olefins, or a combination comprising at least one of the foregoing from the reactor; optionally passing the outlet stream through one or more condensers to a condensate vessel, wherein the outlet stream is split into a vapor fraction and a liquid fraction within the condensate vessel, wherein the vapor fraction comprises greater than or equal to 55 weight percent ethylene and the liquid fraction comprises ethylene, butene-1, solvent, optionally other and/or higher carbon number linear alpha olefins, or a combination comprising at least one of the foregoing; withdrawing the vapor fraction from the condensate vessel, optionally passing the vapor fraction through a purge stream, and optionally passing at least a portion of the vapor fraction through a heat exchanger and recycling it back to the feed stream at a constant flow rate; and withdrawing the liquid fraction from the condensate vessel and injecting it into the reactor at a constant flow rate, wherein the liquid fraction is injected into the liquid phase and/or the vapor phase of the reactor.

These and other features and characteristics are more particularly described below.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 is an ethylene loop around a bubble column reactor.

### DETAILED DESCRIPTION

Described herein is a method for temperature control in a bubble column reactor. The oligomerization, e.g., trimerization, of ethylene into 1-hexene in a bubble column reactor is highly exothermic. The heat generated during the production of 1-hexene needs to be released in order to maintain the reactor at the desired reactor temperature. For example, during the selective production of 1-hexene, the main bubble reactor should desirably be maintained at a reactor temperature of 40-100° C., desirably 40-80° C., more desirably 60-80° C. To achieve maintenance of the desired temperature, the method disclosed herein utilizes two cooling agents advantageous for temperature control during this process.

Ethylene trimerization to 1-hexene can take place in a bubble column reactor. Ethylene feed can bubble through a sparger plate and flow upward in a liquid solvent creating a two phased bed referred to as a two phase level. The solvent can be a single compound or a mixture selected from aromatic solvents or aliphatic solvents. For example, the solvent can comprise toluene, benzene, ethylbenzene, cumene, xylene, mesitylene, hexane, octane, cyclohexane, olefins, such as hexene, heptane, or octane, ethers, such as, diethylether or tetrahydrofuane, or a combination comprising at least one of the foregoing. Aromatic solvents are especially desirable, such as toluene. The reactor overhead outlet stream can be a mixture of unconverted ethylene, butene-1, 1-hexene, solvent, and optionally, traces of other and/or higher carbon number linear alpha olefins, or a combination comprising at least one of the foregoing. The outlet stream can be taken from a gas phase within the reactor. The outlet stream can be partially condensed with one or more partial condensers and sent to a condensate vessel where vapor and liquid phases are separated. Different condensers can be used, for example, in series. For example, the condensers can be operated at different temperature levels. For example, the condensers can include cooling water, propylene as a coolant, crossflow heat exchangers with outer process streams. The vapor phase, e.g., ethylene, can be compressed and/or recycled back to the reactor. Before recycling to the reactor, the vapor fraction can be optionally passed through a purge stream. The purge stream can assist in removing any accumulated trace impurities present in the vapor phase. The liquid condensate, which can contain ethylene, butene-1, solvent, optionally other and/or higher carbon number (e.g., heavier) linear alpha olefins, or a combination comprising at least one of the foregoing can be recycled back to the reactor as a cooling agent.

It was unexpectedly discovered hereof that a combination of two cooling agents can be advantageous in maintaining the required temperature of the bubble column reactor. Advantageous cooling agents can include, for example, the excess ethylene feed and the liquid reflux comprised of light components where the latent heat of vaporization can be employed to remove the heat of reaction.

The first cooling agent can be a flow of liquid condensate that can be routed to the reactor and can cool the reactor using the latent heat of vaporization. The liquid condensate can be recovered directly from the reactor output. The liquid condensate can also be recovered following further processing of the reactor output, for example a condenser and a condensate vessel following reactor output. The liquid condensate can include, for example, ethylene, butene-1, optionally other and/or higher carbon number (e.g., heavier) linear alpha olefins, solvent, or a combination comprising at least one of the foregoing. The liquid condensate can be injected into the reactor, for example, in the gas phase, two phase level, or both. For vapor phase injection, it is contemplated that a distribution system and/or a spray system can optionally be present. The liquid condensate can be injected into the reactor at a constant flow rate. The liquid condensate can cool the reactor via the latent heat of vaporization.

The second cooling agent can be excess ethylene. The ethylene can be fed into the reactor input at a specified inlet temperature at a constant flow rate. Ethylene can be recovered by withdrawing the vapor fraction from the condensate vessel and recycling it back into the reactor input, or the ethylene can be injected directly into the reactor. The vapor fraction of the condensate vessel can comprise greater than or equal to 55 weight percent (wt. %) ethylene, for example, greater than or equal to 60 wt. % ethylene, for example, greater than or equal to 65 wt. % ethylene, for example, greater than or equal to 70 wt. % ethylene, for example, greater than or equal to 75 wt. % ethylene. The vapor fraction can be recycled into the reactor inlet at a constant flow rate. The vapor reaction can be passed through a compressor prior to recycling back to the reactor inlet. The vapor fraction can be passed through a heat exchanger prior to recycling back to the reactor inlet.

The temperature of the ethylene in the reactor inlet can be controlled by mixing hot and cold ethylene to obtain the desired temperature of the inlet. A temperature controller can be included on the inlet into the reactor. The temperature controller can adjust the cold or hot ethylene streams, respectively, to adjust the inlet temperature into the reactor. The temperature controller can be in communication with both the reactor and the vapor fraction input to maintain the reactor temperature in the required range. The temperature controller can be a conventional temperature controller known by those of skill in the art.

1-Hexene is commonly manufactured by two general routes: (i) full-range processes via the oligomerization of ethylene and (ii) on-purpose technology. A minor route to 1-hexene, used commercially on smaller scales, is the dehydration of hexanol. Prior to the 1970s, 1-hexene was also manufactured by the thermal cracking of waxes. Linear internal hexenes were manufactured by chlorination/dehydrochlorination of linear paraffins.

“Ethylene oligomerization” combines ethylene molecules to produce linear alpha-olefins of various chain lengths with an even number of carbon atoms. This approach results in a distribution of alpha-olefins. Oligomerization of ethylene can produce 1-hexene.

Fischer-Tropsch synthesis to make fuels from synthesis gas derived from coal can recover 1-hexene from the aforementioned fuel streams, where the initial 1-hexene concentration cut can be 60% in a narrow distillation, with the remainder being vinylidenes, linear and branched internal olefins, linear and branched paraffins, alcohols, aldehydes, carboxylic acids, and aromatic compounds. The trimerization of ethylene by homogeneous catalysts has been demonstrated.

There are a wide range of applications for linear alpha olefins. The lower carbon numbers, 1-butene, 1-hexene and 1-octene can be used as comonomers in the production of polyethylene. High density polyethylene (HDPE) and linear low density polyethylene (LLDPE) can use approximately 2-4% and 8-10% of comonomers, respectively.

Another use of C₄-C₈ linear alpha olefins can be for production of linear aldehyde via oxo synthesis (hydroformylation) for later production of short-chain fatty acid, a carboxylic acid, by oxidation of an intermediate aldehyde, or linear alcohols for plasticizer application by hydrogenation of the aldehyde.

An application of 1-decene is in making polyalphaolefin synthetic lubricant base stock (PAO) and to make surfactants in a blend with higher linear alpha olefins.

C₁₀-C₁₄ linear alpha olefins can be used in making surfactants for aqueous detergent formulations. These carbon numbers can be reacted with benzene to make linear alkyl benzene (LAB), which can be further sulfonated to linear alkyl benzene sulfonate (LABS), a popular relatively low cost surfactant for household and industrial detergent applications.

Although some C₁₄ alpha olefin can be sold into aqueous detergent applications, C₁₄ has other applications such as being converted into chloroparaffins. A recent application of C₁₄ is as on-land drilling fluid base stock, replacing diesel or kerosene in that application. Although C₁₄ is more expensive than middle distillates, it has a significant advantage environmentally, being much more biodegradable and in handling the material, being much less irritating to skin and less toxic.

C₁₆-C₁₈ linear olefins find their primary application as the hydrophobes in oil-soluble surfactants and as lubricating fluids themselves. C₁₆-C₁₈ alpha or internal olefins are used as synthetic drilling fluid base for high value, primarily off-shore synthetic drilling fluids. The preferred materials for the synthetic drilling fluid application are linear internal olefins, which are primarily made by isomerizing linear alpha-olefins to an internal position. The higher internal olefins appear to form a more lubricious layer at the metal surface and are recognized as a better lubricant. Another application for C₁₆-C₁₈ olefins is in paper sizing. Linear alpha olefins are, once again, isomerized into linear internal olefins and are then reacted with maleic anhydride to make an alkyl succinic anhydride (ASA), a popular paper sizing chemical.

C₂₀-C₃₀ linear alpha olefins production capacity can be 5-10% of the total production of a linear alpha olefin plant. These are used in a number of reactive and non-reactive applications, including as feedstocks to make heavy linear alkyl benzene (LAB) and low molecular weight polymers used to enhance properties of waxes.

The use of 1-hexene can be as a comonomer in production of polyethylene. High-density polyethylene (HDPE) and linear low-density polyethylene (LLDPE) use approximately 2-4% and 8-10% of comonomers, respectively.

Another use of 1-hexene is the production of the linear aldehyde heptanal via hydroformylation (oxo synthesis). Heptanal can be converted to the short-chain fatty acid heptanoic acid or the alcohol heptanol.

A method of temperature control can include passing a feed stream through a reactor at a feed location. The feed stream can comprise ethylene and catalyst. An outlet stream can be withdrawn from the reactor, wherein the outlet stream can comprise linear alpha olefins. The outlet stream can be free from catalyst. The outlet stream can be taken from a vapor phase within the reactor. The outlet stream can be passed through a condensate vessel with the outlet stream being split into a vapor fraction and a liquid fraction within the condensate vessel. The vapor fraction can be removed from the condensate and can be recycled back to the feed stream. Optionally, the vapor fraction can be passed through a purge stream before recycling to the feed stream. The liquid fraction can be withdrawn from the condensate vessel and injected into a reactor at an injection location.

The method can include passing the feed stream through a sparger plate within the reactor. An oligomerization reaction, e.g., a trimerization reaction, can then occur in the reactor. Although described herein with respect to a bubble column reactor, it is to be understood that reactors other than a bubble column reactor can be utilized. A second feed stream can comprise a liquid solvent selected from a single compound or a mixture of aromatic or aliphatic solvents, for example, toluene, benzene, ethylbenzene, cumene, xylenes, mesitylene, hexane, octane, cyclohexane, olefins, such as hexene, heptane, octane, or ethers, such as diethylether or tetrahydrofurane. Desirably, the liquid solvent comprises an aromatic solvent, such as toluene. The second feed stream can be sent to the reactor. The feed stream can comprise toluene in addition to the ethylene. The outlet stream can comprise unconverted ethylene, 1-hexene, butene-1, solvent, optionally other and/or higher carbon number linear alpha olefins, or a combination comprising at least one of the foregoing.

The method can include passing the outlet stream through one or more partial condensers before passing through the condensate vessel. The vapor fraction can comprise greater than or equal to 55 wt. % of ethylene and can be recycled back to the reactor at a constant flow rate. For example, the vapor fraction can be passed through a compressor before it is recycled back to the reactor. The liquid fraction can comprise ethylene, butene-1, solvent, optionally other and/or higher carbon number linear alpha olefins, or a combination comprising at least one of the foregoing. The liquid fraction can be injected into the reactor at a constant flow rate. The reactor can be a two-phase reactor with a liquid phase and a vapor phase. The liquid fraction can be injected into the liquid phase and/or the vapor phase of reactor. For vapor phase injection, a distribution system and/or a spray system can optionally be present. The liquid fraction can cool the reactor via the latent heat of vaporization. The method can further include passing at least a portion of the vapor fraction through a heat exchanger prior to passing through the reactor. A temperature controller can be in communication with the reactor and the vapor fraction. A temperature within the reactor can be maintained at 40° C. to 100° C., for example, 40° C. to 80° C., for example, 40° C. to 60° C.

A method of temperature control as disclosed herein can include passing a feed stream comprising ethylene and a liquid solvent, through a reactor at a feed location, wherein the reactor is a two-phase bubble column reactor comprising a liquid phase and a vapor phase, wherein an oligomerization reaction, such as a trimerization reaction occurs within the reactor. An outlet steam comprising unconverted ethylene, butene-1, solvent, optionally other and/or higher carbon number linear alpha olefins, or a combination comprising at least one of the foregoing can be withdrawn from the reactor. The outlet stream can optionally be passed through one or more condensers to a condensate vessel. The outlet stream can be split into a vapor fraction and a liquid fraction within the condensate vessel, wherein the vapor fraction comprises greater than or equal to 55 wt. % ethylene, for example, greater than or equal to 60 wt. %, for example, greater than or equal to 65 wt. %, for example, greater than or equal to 70 wt. %, for example, greater than or equal to 75 wt. %, and the liquid fraction comprises ethylene, butene-1, solvent, optionally other and/or higher carbon number (e.g., heavier) linear alpha olefins, or a combination comprising at least one of the foregoing. A vapor fraction can be withdrawn from the condensate vessel. The vapor fraction can optionally be passed through a purge stream and at least a portion of the vapor fraction can be passed through a heat exchanger and recycled back to the feed stream at a constant flow rate. The liquid fraction can be withdrawn from the condensate vessel and injecting it into the reactor at a constant flow rate, wherein the liquid fraction is injected into the liquid phase and/or the vapor phase of the reactor.

A more complete understanding of the components, processes, and apparatuses disclosed herein can be obtained by reference to the accompanying drawings. These FIGURES (also referred herein as “FIG.”) are merely schematic representations based on convenience and the ease of demonstrating the present disclosure, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

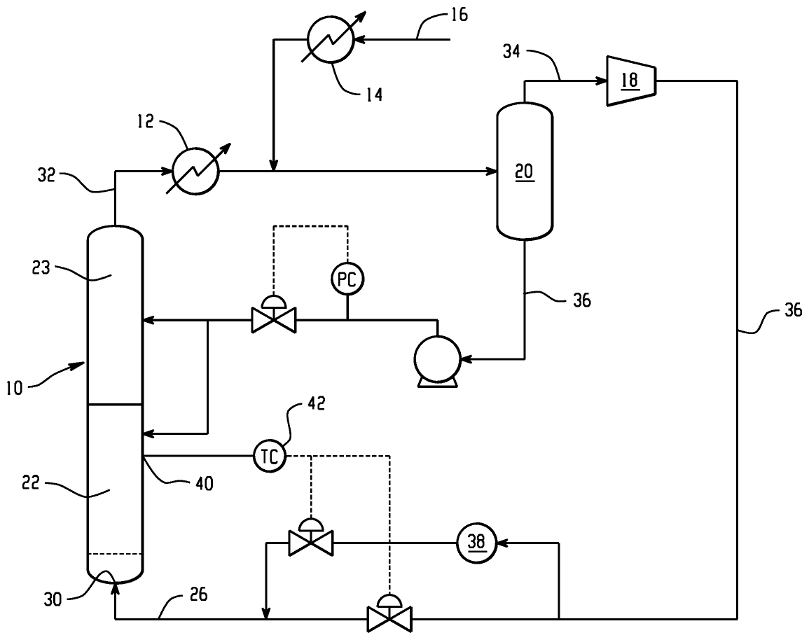

FIG. 1 shows an ethylene loop around bubble column reactor. In FIG. 1, a feed stream 26 is passed through a reactor 10 at a feed location 30. The feed stream 26 can comprise ethylene and catalyst. An outlet stream 32 can be withdrawn from the reactor 10. The outlet stream 32 can comprise linear alpha olefins. The outlet stream 32 can be taken from a vapor phase within the reactor. After exiting the partial condenser 12 and before entering the condensate vessel 20, the outlet stream 32 can be mixed with make-up stream 16. Make-up stream 16 can pass through a chiller 14 before mixing with the outlet stream 32. The feed stream 26 and the make-up stream 16 can independently comprise ethylene. The outlet stream 32 can be passed through a partial condenser 12 and mixed with condensate vessel 20 with the outlet stream 32 being split into a vapor fraction 34 and a liquid fraction 36 within the condensate vessel 20. Optionally, the outlet stream 32 can be passed through one or more partial condensers before passing through the condensate vessel 20. The outlet stream 32 can be free of catalyst. The vapor fraction 34 can be withdrawn from the condensate vessel 20, passed through a compressor 18, and recycled back to the feed stream 26. Optionally, the vapor fraction 34 can be passed through a purge stream. Optionally, the vapor fraction 34 can be passed through a heat exchanger 38 and injected into the reactor 10 at an injection location 40. Optionally, the feed stream 26 can be passed through a sparger plate within the reactor 10. The vapor fraction 34 can be recycled back to the reactor 10 at a constant flow rate. The liquid fraction 36 can be removed from the condensate vessel 20 and injected into the reactor 10 at a constant flow rate. The liquid fraction 36 can be injected into the liquid phase 22 and/or the vapor phase 23 of the reactor. The liquid fraction 36 can cool the reactor 10 via latent heat of vaporization. A temperature controller 42 is in communication with the reactor 10 and the vapor fraction 34. A temperature within the reactor is maintained at a temperature of 40° C. to 100° C., for example, 40° C. to 80° C., for example, 40° C. to 60° C.

The following example is merely illustrative of the methods disclosed herein and are not intended to limit the scope hereof. Unless otherwise stated herein, the example was based upon simulations.

### EXAMPLES

### Example 1

The results from process simulation for the numbered streams in FIG. 1 is shown in Table 1.

TABLE 1
Results of process simulation for the temperature control concept

| Stream number | 1 | 2 | 3 | 4 |
|---|---|---|---|---|
| Vapor fraction | 0 | 1 | 1 | 1 |
| Temperature (° C.) | −3.7 | 1.5 | 144.8 | 12.4 |
| Pressure (bar-g) | 30.1 | 30.1 | 30.1 | 30.1 |
| Flow (tonne/hr) | 30.5 | 19.6 | 1.9 | 21.5 |
| Mass fraction | | | | |
| Ethylene | 0.690621 | 0.954912 | 0.954912 | 0.954912 |
| Butene-1 | 0.246081 | 0.032899 | 0.032899 | 0.032899 |
| Hexene-1 | 0.036080 | 0.000623 | 0.000623 | 0.000623 |
| Solvent | 0.020059 | 0.000126 | 0.000126 | 0.000126 |

The methods disclosed herein include(s) at least the following aspects:

Aspect 1: A method of temperature control, comprising: passing a feed stream comprising ethylene and catalyst through a reactor at a feed location; withdrawing an outlet steam comprising linear alpha olefins from the reactor, wherein the outlet stream is taken from a vapor phase within the reactor; passing the outlet stream through a condensate vessel, wherein the outlet stream is split into a vapor fraction and a liquid fraction within the condensate vessel; withdrawing the vapor fraction from the condensate vessel, optionally passing the vapor fraction through a purge stream, and recycling the purge stream back to the feed stream; and withdrawing the liquid fraction from the condensate vessel and injecting it into the reactor at an injection location.

Aspect 2: The method of Aspect 1, wherein a second feed stream comprises a liquid solvent selected from a single compound or a mixture of aromatic or aliphatic solvents, preferably toluene, benzene, ethylbenzene, cumene, xylenes, mesitylene, hexane, octane, cyclohexane, olefins, preferably, hexene, heptane, octane, or ethers, preferably diethylether or tetrahydrofurane, more preferably an aromatic solvent, most preferably toluene, and wherein the second feed stream is sent to the reactor.

Aspect 3: The method of any of the preceding aspects, further comprising passing the feed stream through a sparger plate within the reactor.

Aspect 4: The method of any of the preceding aspects, wherein the outlet stream is free of catalyst.

Aspect 5: The method of any of the preceding aspects, wherein the reactor is a bubble column reactor.

Aspect 6: The method of any of the preceding aspects, wherein a trimerization reaction occurs within the reactor.

Aspect 7: The method of any of the preceding aspects, wherein the outlet stream comprises unconverted ethylene, 1-hexene, butene-1, solvent, optionally other and/or higher carbon number linear alpha olefins, or a combination comprising at least one of the foregoing.

Aspect 8: The method of any of the preceding aspects, further comprising passing the outlet stream through one or more partial condensers prior to passing through the condensate vessel.

Aspect 9: The method of any of the preceding aspects, wherein the vapor fraction comprises greater than or equal to 55 weight % ethylene, preferably, greater than or equal to 60 weight % ethylene, more preferably, greater than or equal to 65 weight percent ethylene, even more preferably, greater than or equal to 70 weight % ethylene, most preferably 75 weight % ethylene.

Aspect 10: The method of any of the preceding aspects, wherein the vapor fraction is recycled back to the reactor at a constant flow rate.

Aspect 11: The method of any of the preceding aspects, wherein the liquid fraction comprises ethylene, butene-1, solvent, optionally other and/or higher carbon number linear alpha olefins, or a combination comprising at least one of the foregoing.

Aspect 12: The method of any of the preceding aspects, wherein the liquid fraction is injected into the reactor at a constant flow rate.

Aspect 13: The method of any of the preceding aspects, further comprising passing the vapor fraction through a compressor prior to recycling back to the reactor.

Aspect 14: The method of any of the preceding aspects, wherein the reactor is a two-phase reactor, wherein the two-phases are a liquid phase and a vapor phase.

Aspect 15: The method of Aspect 13 or Aspect 15, wherein the liquid fraction is injected into the liquid phase and/or the vapor phase of the reactor, wherein for the vapor phase injection a distribution system and/or spray system is optionally present.

Aspect 16: The method of any of the preceding aspects, wherein the liquid fraction cools the reactor via the latent heat of vaporization.

Aspect 17: The method of any of the preceding aspects, further comprising passing at least a portion of the vapor fraction through a heat exchanger prior to passing through the reactor.

Aspect 18: The method of any of the preceding aspects, wherein a temperature controller is in communication with the reactor and the vapor fraction.

Aspect 19: The method of any of the preceding aspects, wherein a temperature within the reactor is maintained at 40° C. to 100° C.

### Embodiment 20

A method of temperature control, comprising: passing a feed stream comprising ethylene and a liquid solvent through a reactor at a feed location, wherein the reactor is a two-phase bubble column reactor comprising a liquid phase and a vapor phase, wherein an oligomerization reactor, preferably a trimerization reaction occurs within the reactor; withdrawing an outlet steam comprising unconverted ethylene, butene-1, solvent, optionally other and/or higher carbon number linear alpha olefins, or a combination comprising at least one of the foregoing from the reactor; passing the outlet stream through a condensate vessel, wherein the outlet stream is split into a vapor fraction and a liquid fraction within the condensate vessel, wherein the vapor fraction comprises greater than or equal to 55% ethylene and the liquid fraction comprises ethylene, butene-1, solvent, optionally other and/or higher carbon number linear alpha olefins, or a combination comprising at least one of the foregoing; withdrawing the vapor fraction from the condensate vessel, optionally passing the vapor fraction through a purge stream, passing at least a portion of the vapor fraction through a heat exchanger and recycling it back to the feed stream at a constant flow rate; and withdrawing the liquid fraction from the condensate vessel and injecting it into the reactor at a constant flow rate, wherein the liquid fraction is injected into the liquid phase and/or the vapor phase of the reactor.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

Unless otherwise specified herein, any reference to standards, regulations, testing methods and the like, such as ASTM D1003, ASTM D4935, ASTM 1746, FCC part 18, CISPR11, and CISPR 19 refer to the standard, regulation, guidance or method that is in force at the time of filing of the present application.

As used herein, the term “hydrocarbyl” and “hydrocarbon” refers broadly to a substituent comprising carbon and hydrogen, optionally with 1 to 3 heteroatoms, for example, oxygen, nitrogen, halogen, silicon, sulfur, or a combination thereof; “alkyl” refers to a straight or branched chain, saturated monovalent hydrocarbon group; “alkylene” refers to a straight or branched chain, saturated, divalent hydrocarbon group; “alkylidene” refers to a straight or branched chain, saturated divalent hydrocarbon group, with both valences on a single common carbon atom; “alkenyl” refers to a straight or branched chain monovalent hydrocarbon group having at least two carbons joined by a carbon-carbon double bond; “cycloalkyl” refers to a non-aromatic monovalent monocyclic or multicylic hydrocarbon group having at least three carbon atoms, “cycloalkenyl” refers to a non-aromatic cyclic divalent hydrocarbon group having at least three carbon atoms, with at least one degree of unsaturation; “aryl” refers to an aromatic monovalent group containing only carbon in the aromatic ring or rings; “arylene” refers to an aromatic divalent group containing only carbon in the aromatic ring or rings; “alkylaryl” refers to an aryl group that has been substituted with an alkyl group as defined above, with 4-methylphenyl being an exemplary alkylaryl group; “arylalkyl” refers to an alkyl group that has been substituted with an aryl group as defined above, with benzyl being an exemplary arylalkyl group; “acyl” refers to an alkyl group as defined above with the indicated number of carbon atoms attached through a carbonyl carbon bridge (—C(═O)—); “alkoxy” refers to an alkyl group as defined above with the indicated number of carbon atoms attached through an oxygen bridge (—O—); and “aryloxy” refers to an aryl group as defined above with the indicated number of carbon atoms attached through an oxygen bridge (—O—).

Unless otherwise indicated, each of the foregoing groups can be unsubstituted or substituted, provided that the substitution does not significantly adversely affect synthesis, stability, or use of the compound. The term “substituted” as used herein means that at least one hydrogen on the designated atom or group is replaced with another group, provided that the designated atom's normal valence is not exceeded. When the substituent is oxo (i.e., ═O), then two hydrogens on the atom are replaced. Combinations of substituents and/or variables are permissible provided that the substitutions do not significantly adversely affect synthesis or use of the compound. Exemplary groups that can be present on a “substituted” position include, but are not limited to, cyano; hydroxyl; nitro; azido; alkanoyl (such as a C₂₋₆ alkanoyl group such as acyl); carboxamido; C₁₋₆ or C₁₋₃ alkyl, cycloalkyl, alkenyl, and alkynyl (including groups having at least one unsaturated linkages and from 2 to 8, or 2 to 6 carbon atoms); C₁₋₆ or C₁₋₃ alkoxys; C₆₋₁₀ aryloxy such as phenoxy; C₁₋₆ alkylthio; C₁₋₆ or C₁₋₃ alkylsulfinyl; C₁₋₆ or C₁₋₃ alkylsulfonyl; aminodi(C₁₋₆ or C₁₋₃)alkyl; C₆₋₁₂ aryl having at least one aromatic rings (e.g., phenyl, biphenyl, naphthyl, or the like, each ring either substituted or unsubstituted aromatic); C₇₋₁₉ arylalkyl having 1 to 3 separate or fused rings and from 6 to 18 ring carbon atoms; or arylalkoxy having 1 to 3 separate or fused rings and from 6 to 18 ring carbon atoms, with benzyloxy being an exemplary arylalkoxy.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of temperature control, comprising:
passing a feed stream comprising ethylene and catalyst through a reactor at a feed location;
withdrawing an outlet steam comprising linear alpha olefins from the reactor, wherein the outlet stream is taken from a vapor phase within the reactor;
passing the outlet stream through a condensate vessel, wherein the outlet stream is split into a vapor fraction and a liquid fraction within the condensate vessel;
withdrawing the vapor fraction from the condensate vessel, optionally passing the vapor fraction through a purge stream, and recycling the vapor fraction back to the feed stream; and
withdrawing the liquid fraction from the condensate vessel and injecting it into the reactor at an injection location.

2. The method of claim 1, wherein a second feed stream comprises a liquid solvent selected from a single compound or a mixture of aromatic or aliphatic solvents, preferably toluene, benzene, ethylbenzene, cumene, xylenes, mesitylene, hexane, octane, cyclohexane, olefins, preferably, hexene, heptane, octane, or ethers, preferably diethylether or tetrahydrofurane, more preferably an aromatic solvent, most preferably toluene, and wherein the second feed stream is sent to the reactor.

3. The method of claim 1, further comprising passing the feed stream through a sparger plate within the reactor.

4. The method of claim 1, wherein the outlet stream is free of catalyst.

5. The method of claim 1, wherein the reactor is a bubble column reactor.

6. The method of claim 1, wherein a trimerization reaction occurs within the reactor.

7. The method of claim 1, wherein the outlet stream comprises unconverted ethylene, 1-hexene, butene-1, solvent, optionally other and/or higher carbon number linear alpha olefins, or a combination comprising at least one of the foregoing.

8. The method of claim 1, further comprising passing the outlet stream through one or more partial condensers prior to passing through the condensate vessel.

9. The method of claim 1, wherein the vapor fraction comprises greater than or equal to 55 weight % ethylene, preferably, greater than or equal to 60 weight % ethylene, more preferably, greater than or equal to 65 weight percent ethylene, even more preferably, greater than or equal to 70 weight % ethylene, most preferably 75 weight % ethylene.

10. The method of claim 1, wherein the vapor fraction is recycled back to the reactor at a constant flow rate.

11. The method of claim 1, wherein the liquid fraction comprises ethylene, butane-1, solvent, optionally other and/or higher carbon number linear alpha olefins, or a combination comprising at least one of the foregoing.

12. The method of claim 1, wherein the liquid fraction is injected into the reactor at a constant flow rate.

13. The method of claim 1, further comprising passing the vapor fraction through a compressor prior to recycling back to the reactor.

14. The method of claim 1, wherein the reactor is a two-phase reactor, wherein the two-phases are a liquid phase and a vapor phase.

15. The method of claim 12, wherein the liquid fraction is injected into the liquid phase and/or the vapor phase of the reactor, wherein for the vapor phase injection a distribution system and/or spray system is optionally present.

16. The method of claim 1, wherein the liquid fraction cools the reactor via the latent heat of vaporization.

17. The method of claim 1, further comprising passing at least a portion of the vapor fraction through a heat exchanger prior to passing through the reactor.

18. The method of claim 1, wherein a temperature controller is in communication with the reactor and the vapor fraction.

19. The method of claim 1, wherein a temperature within the reactor is maintained at 40° C. to 100° C.

20. A method of temperature control, comprising:
passing a feed stream comprising ethylene and a liquid solvent through a reactor at a feed location, wherein the reactor is a two-phase bubble column reactor comprising a liquid phase and a vapor phase, wherein an oligomerization reaction, preferably a trimerization reaction occurs within the reactor;
withdrawing an outlet steam comprising unconverted ethylene, butene-1, solvent, optionally other and/or higher carbon number linear alpha olefins, or a combination comprising at least one of the foregoing from the reactor;
optionally passing the outlet stream through one or more condensers to a condensate vessel, wherein the outlet stream is split into a vapor fraction and a liquid fraction within the condensate vessel, wherein the vapor fraction comprises greater than or equal to 55 weight % ethylene and the liquid fraction comprises ethylene, butene-1, solvent, optionally other and/or higher carbon number linear alpha olefins, or a combination comprising at least one of the foregoing;
withdrawing the vapor fraction from the condensate vessel, optionally passing the vapor fraction through a purge stream, and optionally passing at least a portion of the vapor fraction through a heat exchanger and recycling it back to the feed stream at a constant flow rate; and
withdrawing the liquid fraction from the condensate vessel and injecting it into the reactor at a constant flow rate, wherein the liquid fraction is injected into the liquid phase and/or the vapor phase of the reactor.
